(19) United States
(12) Patent Application Publication
(10) Pub. No.: US 2024/0156100 A1
(43) Pub. Date: May 16, 2024

(54) ODOR AND MICROBIAL MITIGATION SOLUTION

(71) Applicant: Ken W Donovan, Acton, CA (US)

(72) Inventor: Ken W Donovan, Acton, CA (US)

(21) Appl. No.: 17/985,702

(22) Filed: Nov. 11, 2022

(51) Int. Cl. A01N 59/16; A01N 25/24; A01N 59/06; A01N 59/12; A01P 1/00; A61L 2/00; A61L 2/18; A61L 9/05; C02F 1/50

(52) U.S. Cl. CPC A01N 59/16; A01N 25/24; A01N 59/06; A01N 59/12; A01P 1/00; A61L 2/0088; A61L 2/18; A61L 9/05; C02F 1/50; A61L 2101/30; A61L 2202/25; A61L 2209/21

(57) ABSTRACT

A method for treating foul odors. A chemical solution is administered to the area to be treated. The chemical solution includes: potassium iodine, zinc sulfate and magnesium sulfate. In a preferred embodiment the chemical solution is diluted in water and applied to an area needing treatment.

The present invention relates to chemical solutions, and in particular, to chemical solutions for mitigating and eliminating unwanted odors.

### BACKGROUND OF THE INVENTION

Odor mitigation is a continuous challenge for society. Unwanted odors may be found as a result of microorganisms, bacterium, mold, rotten food, animal excrement, urine, smoke smell, plant smells, cannabis smells and many other sources as well. It is a challenge to mitigate or dispense of unwanted odors. For example, using a bleach or an aggressive cleaner may dispense of the odor, but such powerful chemicals may also damage the surrounding area or kill important plants or harm humans and animals. There are examples in the prior art of odor mitigating solutions. But many of these have drawbacks in that they are harmful to the environment, and/or very expensive and complicated to apply.

What is needed is a safe, non-toxic, non-allergenic, economical, ecological solution to treat the ever-increasing industrial contaminants, toxic gasses and offensive odors in our environment.

### SUMMARY OF THE INVENTION

The present invention provides a method for treating foul odors. A chemical solution is administered to the area to be treated. The chemical solution includes: potassium iodine, zinc sulfate and magnesium sulfate. In a preferred embodiment the chemical solution is diluted in water and applied to an area needing treatment.

### DETAILED DESCRIPTION OF THE PREFERRED EMBODIMENTS

Through several years of development, beta testing and trials, Applicant has created a non-toxic, natural, environmentally friendly, cost-effective H₂O/Iodine based chemical solution that has antimicrobial, anti-odor, oxidation and disinfectant properties. Efficacy of the disinfectant and antimicrobial properties of the chemical solution make it an excellent media for topical cleansing, irrigating and flushing foreign matter from wounds. It is a non-caustic formula containing essential elements that promotes healing and elimination of unpleasant, unwanted odor from an area.

Through experimentation Applicant has discovered that the chemical solution may be applied to volatile organic compounds (VOCs), terpenes, triglycerides, hydrocarbons, inorganic compounds, hydrogen sulfide, natural odors, man-made odors, and ammonia. In a preferred embodiment the chemical solution is a water-based solution that includes potassium iodine and transitional and essential elements. The chemical solution when applied creates a catalytic chemical reaction that oxidizes complex molecules into their base elements, thereby eliminating odors and toxins associated with these compounds.

Further benefits of the chemical solution include the elimination of microorganisms, bacteria and fungi when applied as directed. This benefit continues until the oxidation properties of the chemistry has been depleted, the remaining iodate and essential elements promote healing and growth.

In preferred embodiments, a combination of the following is utilized to create a preferred chemical solution to eliminate odors and act as a disinfectant: Water (H₂O), Saline (H2O) (NaCl), Potassium Iodine (KI), Zinc Sulfate (ZnSO4), Manganese Sulfate (MnSO4), Sulfamic Acid (H₃NSO3), Sodium Bicarbonate (NaHCO2), and Sodium Polyacrylate (C3H3NaO2). It should be noted that all components of the chemical solution are designated by the FDA as GRAS (generally recognized as safe) under the conditions and concentrations of its intended use. Also, Sulfamic Acid and Sodium Bicarbonate are inert additives to raise or lower the pH of the solution to a neutral/non irritating 7.0 to 7.4. Also, it should be noted that Saline can be substituted for H₂O.

Applicant has determined through experimentation that the following preferred embodiments are each very effective to create a preferred chemical solution to eliminate odors and act as a disinfectant:
1. H2O, 600 ppm Potassium Iodine, 300 ppm Zinc Sulfate, 300 ppm Manganese Sulfate.
2. H2O, 600 ppm Potassium Iodine, 300 ppm Zinc Sulfate, 300 ppm Manganese Sulfate,
Sulfamic Acid (as needed to lower pH).

3. H2O, 600 ppm Potassium Iodine, 300 ppm Zinc Sulfate, 300 ppm Manganese Sulfate, Sodium Bicarbonate (as needed to raise pH).
4. H2O, 600 ppm Potassium Iodine, 300 ppm Zinc Sulfate, 300 ppm Manganese Sulfate, 6250 ppm Sodium Polyacrylate (gelling agent)
5. H2O, 200 ppm Potassium Iodine, 100 ppm Zinc Sulfate, 100 ppm Manganese Sulfate, 6250 ppm Sodium Polyacrylate (gelling agent), Sulfamic Acid (as need to lower pH).
6. H2O, 200 ppm Potassium Iodine, 100 ppm Zinc Sulfate, 100 ppm Manganese Sulfate, 6250 ppm Sodium Polyacrylate (gelling agent), Sodium Bicarbonate (as needed to raise pH).

Additional Findings Determined Efficacy of the chemical solution has been demonstrated from full strength formula to a dilution ratio of up to 200:1 (H2O/Chemistry). Formulas can be super concentrated and remain in solution in ratios more than 250:1 (chemistry: H2O). Formulas can be manufactured and packaged in dry form and added to H2O by the end user, mixing on site into the ready to use solution. As an aqueous solution, the chemical solution can be used in an extensive variety of environments. It can be applied directly to surfaces, countertops, tables, floors, walls, ceilings, animals, bedding, clothes, and laundry, for example. When used as an aerosol it completely eliminates airborne household odors, pet odors, cooking odors, bathroom odors, trash and waste odors, odors from manufacturing processes. A solution that can be deployed into the atmosphere as an atomized aqueous aerosol. The atomized H2O chemistry plays a major role in scavenging airborne pollutants. Reactions occur on the thin water films in the atmospheric aerosols.

The Langmuir—Hinshelwood reaction mechanism was utilized to qualify the reaction rate and thermodynamics of absorption and oxidation of organic molecules on the aqueous aerosol surface. Preferred atomization methods of the chemical solution include: 1) a hand spray bottle, 2) a hydraulic high-pressure pump through a high-pressure nozzle, and 3) a pneumatic medium or low-pressure media delivery nozzle.

It should be further noted that the chemical solution can be applied with direct application of the liquid or gel chemistry to the affected area by hand squeeze bottle. Also, the chemical solution can be applied with direct application of the liquid or gel chemistry to the affected area by sterile compress. Also, the solution can be applied with direct application of the liquid or gel chemistry to the affected area by a wash bottle with dispensing nozzle.

In another preferred embodiment the chemical solution is utilized to treat odor from well water. A naturally occurring problem with well water is hydrogen sulfide gas (H2S) giving the water a “rotten egg” smell and taste. The chemical solution can be poured directly into the water or injected into the water stream eliminating the offensive odors, taste, bacteria, organic and inorganic compounds and other contaminants.

Although the above-preferred embodiments have been described with specificity, persons skilled in this art will recognize that many changes to the specific embodiments disclosed above could be made without departing from the spirit of the invention. For example, it was described above how the above-described chemical solution may be applied to an area for disinfecting the area and for treating foul odors that are in the area, such as a well containing well water. Listed below are further examples of areas that include environments and operations that would show beneficial results with the utilization of the chemical solution. The below list includes only some of the areas the chemical solution may be applied. Many other areas are also possible. Preferred areas include: homes and offices, automobiles, animal care (i.e., veterinary offices, mobile veterinarians, animal groomers), cannabis and CBD, medical facilities (i.e., doctor offices, hospitals, dentist offices, chiropractic offices, pharmacies), ranching, farming, dairy, agriculture, fertilizer (i.e., manufacturing and application), pesticides (i.e., (manufacturing and application), water treatment, waste water treatment, recycling centers, landfills, waste hauling, waste handling, waste container treatment, materials recovery facilities, manufacturing facilities, super fund cleanup sites, power plants, foundries, bakeries, painting facilities (i.e., paints and coatings), food processing facilities, animal food processing facilities, stadiums, parks, hotels, motels, restaurants, military facilities, smoke (cigarette smoke and residue), airplanes, and boats. Therefore, the attached claims and their legal equivalents should determine the scope of the invention.

What is claimed is:

1. A method of treating foul odors and disinfecting comprising the steps of administering to an area to be treated a chemical solution, wherein said chemical solution comprises a mixture of:
A. potassium iodine,
B. zinc sulfate, and
C. magnesium sulfate.

2. The method as in claim 1 wherein said chemical solution is diluted in water.

3. The method as in claim 1, wherein said chemical solution is diluted in water at approximately 600 ppm potassium iodine, 300 ppm zinc sulfate and 300 ppm magnesium sulfate.

4. The method as in claim 1, wherein said chemical solution is diluted in water at approximately 600 ppm potassium iodine, 300 ppm zinc sulfate and 300 ppm magnesium sulfate, and further comprising Sulfamic Acid to lower pH.

5. The method as in claim 1, wherein said chemical solution is diluted in water at approximately 600 ppm potassium iodine, 300 ppm zinc sulfate and 300 ppm magnesium sulfate, and further comprising Sodium Bicarbonate to raise pH.

6. The method as in claim 1, wherein said chemical solution is diluted in water at approximately 600 ppm potassium iodine, 300 ppm zinc sulfate and 300 ppm magnesium sulfate, and further comprising 6250 ppm Sodium Polyacrylate to act as a gelling agent.

7. The method as in claim 1, wherein said chemical solution is diluted in water at approximately 200 ppm potassium iodine, 100 ppm zinc sulfate and 100 ppm magnesium sulfate, and further comprising:
A. 6250 ppm Sodium Polyacrylate to act as a gelling agent, and
B. Sulfamic Acid to lower pH.

8. The method as in claim 1, wherein said chemical solution is diluted in water at approximately 200 ppm potassium iodine, 100 ppm zinc sulfate and 100 ppm magnesium sulfate, and further comprising:
A. 6250 ppm Sodium Polyacrylate to act as a gelling agent, and
B. Sodium Bicarbonate to raise pH.

9. The method as in claim 1, wherein said chemical solution is applied to physical area needing treatment.

10. The method as in claim 1, wherein said chemical solution is applied to at least one plant needing treatment.

11. The method as in claim 1, wherein said chemical solution is applied to at least one animal needing treatment.

12. The method as in claim 1, wherein said chemical solution is applied to at least one human needing treatment.

13. The method as in claim 1, wherein said chemical solution is dispensed into atmosphere needing treatment.

14. The method as in claim 1, wherein said chemical solution is injected into water needing treatment.

15. The method as in claim 1, wherein said chemical solution is applied to a process needing treatment.
